(19) United States
(12) Patent Application Publication
(10) Pub. No.: US 2021/0062256 A1
(43) Pub. Date: Mar. 4, 2021

(54) SYSTEMS AND METHODS FOR NON-INVASIVE PREIMPLANTATION GENETIC DIAGNOSIS

(71) Applicant: CooperGenomics, Inc., Trumbull, CT (US)

(72) Inventors: Santiago MUNNE-BLANCO, Short Hills, NJ (US); Dhruti Ashokbhai BARARIYA, Trumbull, CT (US); Arun Prasad MANOHARAN, Trumbull, CT (US); Dagan WELLS, Oxford (GB)

(21) Appl. No.: 16/644,918

(22) PCT Filed: Sep. 7, 2018
(86) PCT No.: PCT/US2018/049976

§ 371 Date: Mar. 5, 2020

Related U.S. Application Data
(60) Provisional application No. 62/555,466, filed on Sep. 7, 2017.

(51) Int. Cl. C12Q 1/6869; G16B 20/10; G16B 20/20; G16B 30/10

(52) U.S. Cl. CPC C12Q 1/6869; G16B 20/10; G16B 20/20; C12Q 2537/165; C12Q 2521/501; C12Q 2535/122; C12Q 2537/16; G16B 30/10

(57) ABSTRACT

A system for identifying genomic features in an embryo candidate is disclosed. The system includes a genomics sequencer, a computing device and a display. The genomic sequencer is configured to obtain sequence information from concatenated genomic fragments derived from an embryo candidate. The concatenated genomic fragments each contain at least one genomic linker segment and at least one genomic fragment from the embryo candidate. The computing device is communicatively connected to the genomic sequencer and includes a sequence alignment engine and a genomic features identification engine. The sequence alignment engine is configured to subtract out sequence information related to the genomic linker segment portion of the concatenated genomic fragments and align the genomic fragment sequences to a reference genome. The genomic features identification engine is configured to identify genomic features in the aligned genomic fragment sequences. The display is communicatively connected to the computing device and configured to display a report containing the identified genomic features.

### FIELD

The embodiments disclosed herein are generally directed towards systems and methods for non invasive genetic screening and/or diagnosis of embryos prior to implantation in an in vitro fertilization procedure. More specifically, there is a need for non invasive preimplantation screening and/or diagnostic systems and methods which can aid clinicians in the selection of embryos with the lowest risk of genetic abnormalities/defects and have the highest probability of uterine implantation success.

### BACKGROUND

In vitro fertilization (IVF) is an assisted reproductive technology has become increasingly popular for women of advanced maternal age, couples with difficulties conceiving and as a means for facilitating gestational surrogacy. The process of fertilization involves extracting eggs, retrieving a sperm sample, and then manually combining an egg and sperm in a laboratory setting. The embryo(s) is then implanted in the host uterus to carry the embryo to term.

IVF procedures are expensive and can exact a significant emotional/physical toll on patients, so genetic screening of embryos prior to implantation is becoming an increasingly common for patients undergoing an IVF procedure. Current methods of diagnosing genetic abnormalities in embryos and screening for viability of transfer (i.e., embryo implantation viability) require a biopsy of embryos, which can affect embryo quality and requires specialized laboratory techniques that can be prohibitively expensive and time consuming.

As such, there is a need for non-invasive genetic screening and/or diagnostic (NI PGS) systems and methods for genetically screening embryos which avoid the need for embryo biopsy and thus substantially increase the safety for the preimplanted embryo.

### SUMMARY

In one aspect, a method for determining copy number variation in an embryo candidate for in vitro fertilization (IVF) implantation is disclosed. An embryo candidate is isolated from a plurality of embryos. The embryo candidate is incubated in media that is substantially free of DNA. A portion of the media is transferred to an amplification vessel, wherein the portion of media includes genomic fragments shed or secreted from the embryo candidate. A plurality of genomic linker segments and ligase enzyme is added to the amplification vessel in conditions that catalyze the formation of concatenated genomic fragments containing at least one genomic linker segment and at least one genomic fragment from the isolated embryo candidate. The concatenated genomic fragments are amplified in the amplification vessel. Sequence information is obtained from the amplified concatenated genomic fragments. The sequence information is aligned (mapped) against a reference genome. Copy number variations are identified in the embryo candidate when a frequency of genomic fragment sequence reads aligned to a chromosomal position on the reference genome deviates from a frequency threshold.

In another aspect, a method is for identifying genomic features in an embryo candidate is disclosed. An embryo candidate is isolated from a plurality of embryo candidates. The embryo candidate is incubated in media that is substantially free of DNA. A portion of the media is transferred to an amplification vessel, wherein the portion of media includes one more genomic fragments shed or secreted from the embryo candidate. A plurality of genomic linker segments and a ligase enzyme is added to the amplification vessel in conditions that catalyze the formation of concatenated genomic fragments containing at least one genomic linker segment and at least one genomic fragment from the isolated embryo candidate. The concatenated genomic fragments are amplified in the amplification vessel. Sequence information is obtained from the concatenated genomic fragments. The sequence information is aligned against a reference genome. Genomic features are identified on the aligned genomic fragment sequences.

In still another aspect, a system for identifying genomic features in an embryo candidate is disclosed. The system includes a genomics sequencer, a computing device and a display.

The genomic sequencer is configured to obtain sequence information from concatenated genomic fragments derived from an embryo candidate. The concatenated genomic fragments each contain at least one genomic linker segment and at least one genomic fragment from the embryo candidate.

The computing device is communicatively connected to the genomic sequencer and includes a sequence alignment engine and a genomic features identification engine. The sequence alignment engine is configured to subtract out sequence information related to the genomic linker segment portion of the concatenated genomic fragments and align the genomic fragment sequences to a reference genome. The genomic features identification engine is configured to identify genomic features in the aligned genomic fragment sequences. The display is communicatively connected to the computing device and configured to display a report containing the identified genomic features.

In yet another aspect, a method for identifying genomic features in a tissue sample is disclosed. Concatenated genomic fragment sequence reads are received containing at least one genomic linker segment sequence and at least one genomic fragment sequence from a tissue sample. The genomic linker segment sequence portion of the concatenated genomic fragment sequence reads is subtracted out. The concatenated genomic fragment sequence reads are aligned (mapped) to a reference genome. Genomic features are identified on the aligned genomic fragment sequences.

In still another aspect, a non-transitory computer-readable medium is provided in which a program is stored for causing a computer to perform a method for identifying genomic features in a tissue sample. Concatenated genomic fragment sequence reads are received containing at least one genomic linker segment sequence and at least one genomic fragment sequence from a tissue sample. The genomic linker segment sequence portion of the concatenated genomic fragment sequence reads are subtracted out. The concatenated genomic fragment sequence reads are aligned (mapped) to a reference genome. Genomic features are identified on the aligned genomic fragment sequences.

### BRIEF DESCRIPTION OF THE DRAWINGS

For a more complete understanding of the principles disclosed herein, and the advantages thereof, reference is now made to the following descriptions taken in conjunction with the accompanying drawings, in which:

FIG. 1 illustrates a workflow for non-invasive preimplantation genetic screening of embryos, in accordance with some embodiments of the disclosure.

FIG. 2 is an exemplary flowchart depicting an amplification protocol for amplifying short genomic fragments, in accordance with some embodiments of the disclosure.

FIG. 3 illustrates the formation of concatenated fragments, in accordance with some embodiments of the disclosure.

FIG. 4 is a block diagram that illustrates a computer system, in accordance with various embodiments.

FIG. 5 is a schematic diagram of a system for non-invasive preimplantation genetic screening of embryos, in accordance with various embodiments

FIG. 6 is a depiction of how concatenated fragment reads are mapped to a reference genome, in accordance with various embodiments.

FIG. 7 is an exemplary flowchart showing a method for aligning genomic fragment reads to identify various types of genomic features, in accordance with various embodiments.

FIG. 8 is a flowchart showing a method for determining copy number variation in an embryo candidate, in accordance with various embodiments.

FIG. 9 is a flowchart showing a method of identifying genomic features in an embryo candidate, in accordance with various embodiments.

FIG. 10 is a flowchart showing a method for identifying genomic features from concatenated genomic fragment reads, in accordance with various embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

This specification describes exemplary embodiments and applications of the disclosure. The disclosure, however, is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion. In addition, as the terms “on,” “attached to,” “connected to,” “coupled to,” or similar words are used herein, one element (e.g., a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques described herein are those well known and commonly used in the art.

The phrase “next generation sequencing” (NGS) refers to sequencing technologies having increased throughput as compared to traditional Sanger- and capillary electrophoresis-based approaches, for example with the ability to generate hundreds of thousands of relatively small sequence reads at a time. Some examples of next generation sequencing techniques include, but are not limited to, sequencing by synthesis, sequencing by ligation, and sequencing by hybridization. More specifically, the MISEQ, HISEQ and NEXTSEQ Systems of Illumina and the Personal Genome Machine (PGM) and SOLiD Sequencing System of Life Technologies Corp, provide massively parallel sequencing of whole or targeted genomes. The SOLiD System and associated workflows, protocols, chemistries, etc. are described in more detail in PCT Publication No. WO 2006/084132, entitled “Reagents, Methods, and Libraries for Bead-Based Sequencing,” international filing date Feb. 1, 2006, U.S. patent application Ser. No. 12/873,190, entitled “Low-Volume Sequencing System and Method of Use,” filed on Aug. 31, 2010, and U.S. patent application Ser. No. 12/873,132, entitled “Fast-Indexing Filter Wheel and Method of Use,” filed on Aug. 31, 2010, the entirety of each of these applications being incorporated herein by reference thereto.

The phrase “sequencing run” refers to any step or portion of a sequencing experiment performed to determine some information relating to at least one biomolecule (e.g., nucleic acid molecule).

As used herein, the phrase “genomic features” can refer to a genome region with some annotated function (e.g., a gene, protein coding sequence, mRNA, tRNA, rRNA, repeat sequence, inverted repeat, miRNA, siRNA, etc.) or a genetic/genomic variant (e.g., single nucleotide polymorphism/variant, insertion/deletion sequence, copy number variation, inversion, etc.) which denotes a single or a grouping of genes (in DNA or RNA) that have undergone changes as referenced against a particular species or sub-populations within a particular species due to mutations, recombination/crossover or genetic drift.

Genomic variants can be identified using a variety of techniques, including, but not limited to: array-based methods (e.g., DNA microarrays, etc.), real-time/digital/quantitative PCR instrument methods and whole or targeted nucleic acid sequencing systems (e.g., NGS systems, Capillary Electrophoresis systems, etc.). With nucleic acid sequencing, coverage data can be available at single base resolution.

DNA (deoxyribonucleic acid) is a chain of nucleotides consisting of 4 types of nucleotides; A (adenine), T (thymine), C (cytosine), and G (guanine), and that RNA (ribonucleic acid) is comprised of 4 types of nucleotides; A, U (uracil), G, and C. Certain pairs of nucleotides specifically bind to one another in a complementary fashion (called complementary base pairing). That is, adenine (A) pairs with thymine (T) (in the case of RNA, however, adenine (A) pairs with uracil (U)), and cytosine (C) pairs with guanine (G). When a first nucleic acid strand binds to a second nucleic acid strand made up of nucleotides that are complementary to those in the first strand, the two strands bind to form a double strand. As used herein, “nucleic acid sequencing data,” “nucleic acid sequencing information,” “nucleic acid sequence,” “genomic sequence,” “genetic sequence,” or “fragment sequence,” or “nucleic acid sequencing read” denotes any information or data that is indicative of the order of the nucleotide bases (e.g., adenine, guanine, cytosine, and thymine/uracil) in a molecule (e.g., whole genome, whole transcriptome, exome, oligonucleotide, polynucleotide, fragment, etc.) of DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic signature-based systems, etc.

A “polynucleotide”, “nucleic acid”, or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Usually oligonucleotides range in size from a few monomeric units, e.g. 3-4, to several hundreds of monomeric units. Whenever a polynucleotide such as an oligonucleotide is represented by a sequence of letters, such as “ATGCCTG,” it will be understood that the nucleotides are in 5′->3′ order from left to right and that “A” denotes deoxyadenosine, “C” denotes deoxycytidine, “G” denotes deoxyguanosine, and “T” denotes thymidine, unless otherwise noted. The letters A, C, G, and T may be used to refer to the bases themselves, to nucleosides, or to nucleotides comprising the bases, as is standard in the art.

The phrase “fragment library” refers to a collection of nucleic acid fragments, wherein one or more fragments are used as a sequencing template. A fragment library can be generated, for example, by cutting or shearing a larger nucleic acid into smaller fragments. Fragment libraries can be generated from naturally occurring nucleic acids, such as mammalian or bacterial nucleic acids. Libraries comprising similarly sized synthetic nucleic acid sequences can also be generated to create a synthetic fragment library.

In various embodiments, a sequence alignment method can align a fragment sequence to a reference sequence or another fragment sequence. The fragment sequence can be obtained from a fragment library, a paired-end library, a mate-pair library, a concatenated fragment library, or another type of library that may be reflected or represented by nucleic acid sequence information including for example, RNA, DNA, and protein based sequence information. Generally, the length of the fragment sequence can be substantially less than the length of the reference sequence. The fragment sequence and the reference sequence can each include a sequence of symbols. The alignment of the fragment sequence and the reference sequence can include a limited number of mismatches between the symbols of the fragment sequence and the symbols of the reference sequence. Generally, the fragment sequence can be aligned to a portion of the reference sequence in order to minimize the number of mismatches between the fragment sequence and the reference sequence.

In particular embodiments, the symbols of the fragment sequence and the reference sequence can represent the composition of biomolecules. For example, the symbols can correspond to identity of nucleotides in a nucleic acid, such as RNA or DNA, or the identity of amino acids in a protein. In some embodiments, the symbols can have a direct correlation to these subcomponents of the biomolecules. For example, each symbol can represent a single base of a polynucleotide. In other embodiments, each symbol can represent two or more adjacent subcomponent of the biomolecules, such as two adjacent bases of a polynucleotide. Additionally, the symbols can represent overlapping sets of adjacent subcomponents or distinct sets of adjacent subcomponents. For example, when each symbol represents two adjacent bases of a polynucleotide, two adjacent symbols representing overlapping sets can correspond to three bases of polynucleotide sequence, whereas two adjacent symbols representing distinct sets can represent a sequence of four bases. Further, the symbols can correspond directly to the subcomponents, such as nucleotides, or they can correspond to a color call or other indirect measure of the subcomponents. For example, the symbols can correspond to an incorporation or non-incorporation for a particular nucleotide flow.

In various embodiments, a computer program product can include instructions to select a contiguous portion of a fragment sequence; instructions to map the contiguous portion of the fragment sequence to a reference sequence using an approximate string matching method that produces at least one match of the contiguous portion to the reference sequence.

In various embodiments, a system for nucleic acid sequence analysis can include a data analysis unit. The data analysis unit can be configured to obtain a fragment sequence from a sequencing instrument, obtain a reference sequence, select a contiguous portion of the fragment sequence, and map the contiguous portion of the fragment sequence to the reference sequence using an approximate string mapping method that produces at least one match of the contiguous portion to the reference sequence.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

The term “ones” means more than one.

As used herein, the term “plurality” can be 2, 3, 4, 5, 6, 7, 8, 9, 10, or more.

As used herein, the term “cell” is used interchangeably with the term “biological cell.” Non-limiting examples of biological cells include eukaryotic cells, plant cells, animal cells, such as mammalian cells, reptilian cells, avian cells, fish cells, or the like, prokaryotic cells, bacterial cells, fungal cells, protozoan cells, or the like, cells dissociated from a tissue, such as muscle, cartilage, fat, skin, liver, lung, neural tissue, and the like, immunological cells, such as T cells, B cells, natural killer cells, macrophages, and the like, embryos (e.g., zygotes), oocytes, ova, sperm cells, hybridomas, cultured cells, cells from a cell line, cancer cells, infected cells, transfected and/or transformed cells, reporter cells, and the like. A mammalian cell can be, for example, from a human, a mouse, a rat, a horse, a goat, a sheep, a cow, a primate, or the like.

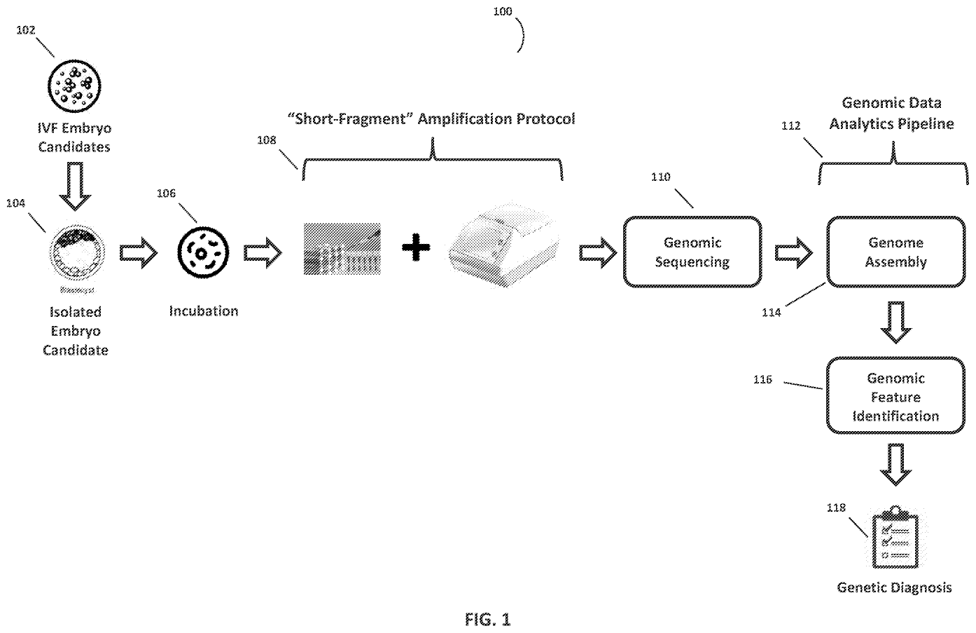

FIG. 1 illustrates a workflow 100 for non-invasive preimplantation genetic screening of embryos, in accordance with some embodiments of the disclosure. As depicted herein, an embryo candidate 104 for IVF implantation can be isolated from a pool of embryos and incubated for a period of time in a sample holder containing media that is substantially free of DNA 106 or other polynucleotides that can interfere with the genetic screening analysis. Some examples of a sample holder may include, but are not limited to, a test tube, pipette tube, petri dish, or a well/partition within a multi-partition/well plate. In various embodiments, the embryo candidate 104 can also be incubated in a continuous culture system whereby “fresh” culture media 106 is introduced using a continuous media feed line to the sample holder and “old” culture media 106 is continuously removed (and sampled) from the sample holder to maintain a substantially constant volume of media in the sample holder.

During incubation, genomic fragments are regularly secreted by and/or shed from the embryo into the surrounding DNA-free media. An example of DNA free media that can be utilized in this workflow is ORIGIO SEQUENTIAL BLAST™ culture media of The Cooper Companies. In some embodiments, the embryo can be incubated in the culture media for a minimum of about 18 hrs. In other embodiments, the embryo can be incubated in the culture media between about 18 hours and about 144 hours. It should be understood that the embryos can be incubated in DNA free media for as long a period of time as is necessary for a sufficient quantity of genomic fragments to be secreted by and/or shed from the embryo to allow for a genetic screening analysis to be performed using the workflow 100. In some embodiments, the embryo is in the blastocyst stage of development when it is isolated and incubated in the DNA free media. In other embodiments, the embryo is in a multi-cell pre-blastocyst stage of development when it is isolated and incubated in the DNA free media.

After the embryo is incubated for a required period of time to allow for a threshold quantity of genomic fragments to be secreted or shed into the DNA free media, a portion of the incubation media is transferred to a separate amplification vessel where the fragments undergo an amplification protocol 108 that is tailored for amplifying short genomic fragment for later genomic sequence analysis. In some embodiments, the amplification protocol 108 uses a multiple displacement amplification (MDA) based whole genome amplification (WGA) technique. MDA is a non-PCR based DNA amplification technique which has been shown to be efficient in the amplification of small amounts of DNA. MDA relies on priming of target DNA with random primers and the use of the strand-displacing φ29 polymerase (or its equivalent) to amplify substantially the entire DNA in a given sample. Compared with PCR-based WGA methods, MDA reduces amplification bias by orders of magnitude, generates longer genomic fragments and exhibits better genome coverage. In other embodiments, the amplification protocol 108 uses a multiple annealing and looping-based amplification cycles (MALBAC) based WGA technique. The MALBAC amplification technique uses special primers that allow amplicons to have complementary ends and therefore to loop, preventing DNA from being copied exponentially. This results in amplification of only the original genomic DNA. This controlled amplification consequently can reduce amplification bias and, by extension, can lower production of artifacts and lower incidences of false positive and false negative mutation calls on the isolated embryo candidate.

It should be understood, however, that any type of WGA technique can be used in amplification protocol 108 as long as the technique generates sufficient quality and/or quantities of genomic fragments to be sequenced for a genetic screening analysis to be run using workflow 100.

After the genomic fragments (from the isolated embryo 104) have been amplified to a sufficient quantity, they are sequenced 110 using a NGS or equivalent genomic sequencing system. The sequencing workflow can begin with the fragments being sequenced 110 on a nucleic acid sequencer to provide hundreds, thousands or millions of nucleic acid sequence reads (i.e., sequence reads). The genomic fragment sequence information can then be processed using a genomic data analytics pipeline 112 whereby the genomic fragment sequences are aligned (mapped) 114 against a reference genome and one or more secondary analytics tools/pipelines are used to help identify one or more genomic features 116 present in the genome of the embryo 104. In some embodiments, the genomic features 116 can be genomic variants such as insertions/deletions (INDEL), copy number variations (CNV), single nucleotide polymorphisms (SNP), duplications, inversions, translocations, etc. In other embodiments, the genomic features 116 can be genomic regions that have some annotated function such as a gene, protein coding sequence, mRNA, tRNA, rRNA, repeat sequence, inverted repeat, miRNA, siRNA, etc. In still other embodiments, the genomic features 116 can be epigenetic changes on the genome (e.g., methylation, acetylation, ubiquitylation, phosphorylation, sumoylation, ribosylation, citrullination, etc.) that can affect gene expression and activity.

In some embodiments, the reference genome is a human genome. In other embodiments, the reference genome is a genome of the animal species that the embryo originates from. It should be appreciated, however, that the reference genome can be an artificially created genome that is not associated with any particular animal species, but rather created for a particular analysis/application.

After the genomic features 116 have been identified, the analytics pipeline 112 can generate a genetic diagnostics report 118 providing information regarding inherited or non-inherited genetic conditions that the isolate embryo 104 has or is at risk for.

In various embodiments, a “blank” or control sample is run side by side with the embyro candidate 104 through the entire workflow 100. That is, a portion of DNA free media (which was not used to incubate an embryo 104) is run through all the steps and processes of workflow 100. The results from analyzing the blank sample can serve as a control to ensure that the genomic features identified in the genome of the embryo is not an artifact of the amplification and/or systemic errors during sequencing.

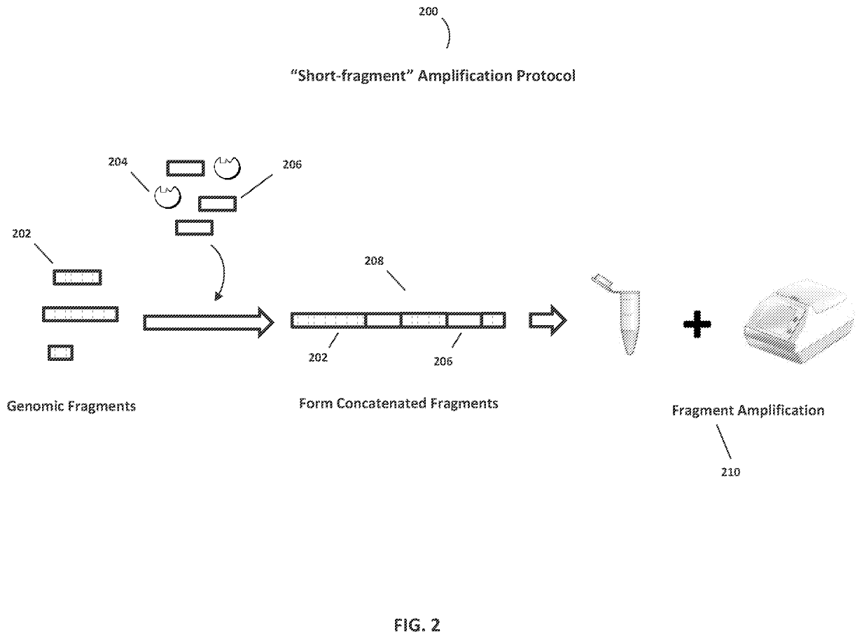

FIG. 2 is an exemplary flowchart depicting an amplification protocol 200 for amplifying short genomic fragments, in accordance with some embodiments of the disclosure.

As depicted herein, the genomic fragments 202 (in the portion of media incubating the embryo) are combined with enzymes 204 and genomic linker segments 206 in conditions that catalyze the formation of concatenated fragments 208. The ligation reaction is carried out at room temperature (without agitation) for about 16-18 hours (overnight incubation). The ligation reaction mixture consists of 1 unit of DNA ligase in a buffer containing 50 mM Tris HCl, 10 mM MgCl₂, 1 mM ATP and 10 mM DTT at a pH of about 7.5 and a temperature of between about 20° C. and about 25° C. temperature. The resulting concatenated fragments 208 are longer than the original genomic fragments 202, which helps to reduce amplification errors (when compared to amplifying the genomic fragments 202 individually) when the genomic fragments are amplified later in the protocol 200.

Concatenation can provide long templates (i.e., concatenated fragments) that are optimal for amplification using the φ29 enzyme, which isothermally amplifies DNA by multiple displacement amplification. φ29 enzyme cannot efficiently and/or accurately amplify short fragments (i.e., amplicons shorter than about 30 base pairs), which has been demonstrated in validation experiments and hence it is pertinent that we create long concatenated fragments to capture the entirety of the short fragments of DNA extruded by the embryo into the culture media. Moreover, concatenation also helps in creating adequate templates for successful amplification by other whole genome amplification strategies such as Sureplex system (Illumina), MALBAC and DOP PCR. This reduction in amplification errors is particularly significant for short genomic fragments. In general, reducing amplification error results in better accuracy in the identification of genomic features when the genomic fragments are later sequenced and analyzed. In some embodiments, the genomic fragment is a short genomic fragment that has a length of between about 30 base pairs (bps) and about 800 bps. In other embodiments, the genomic fragment is a short genomic fragment that has a length of between bout 150 bps to about 400 bps. In still other embodiments, the genomic fragment is a short genomic fragment that has a length of less than about 1000 bps.

The genomic linker segments 206 are essentially artificially created double-stranded “conjoint” oligonucleotide segments of a known length and nucleotide sequence. In some embodiments, the genomic linker segments 206 are between about 30 to 1000 bps in length. In other embodiments, the genomic linker segments 206 are between about 30 bps and about 500 bps in length. In still other embodiments, the genomic linker segments 206 are between about 50 bps to about 150 bps. In some embodiments, the genomic linker segments 206 are homopolymer oligonucleotide segments. In other embodiments, the genomic linker segments 206 are heteropolymer oligonucleotide segments. In some embodiments, the genomic linker segments 206 are blunt ended double-stranded oligonucleotide segments. In some embodiments, the genomic fragments 202 are enzymatically blunt ended prior to being ligated to the genomic linker segments 206.

Various types of prokaryotic and eukaryotic enzymes (i.e., ligases) can be used to ligate the genomic fragments 202 to the genomic linker segments 206 to form the concatenated genomic fragments 208. Some examples of ligases that can be used here include, but are not limited to, T3, T4, T7, or Ligase 1.

After the concatenated fragments are formed in their container (e.g., well, pipette tube, etc.) they can be amplified 210 on a thermal cycler (or similar device) using WGA techniques such as MDA, MALBAC, etc.

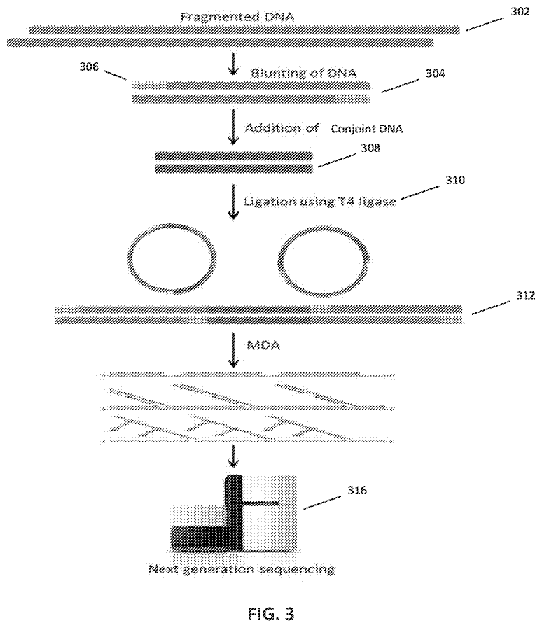

FIG. 3 illustrates the formation of concatenated fragments, in accordance with some embodiments of the disclosure. As depicted herein, the genomic fragments 302 are first blunt ended using a blunting enzyme to fill-in or remove the 3′ or 5′ overhangs (i.e., unpaired nucleotides) 306 prior to the introduction of the genomic linker segments 308 and their ligation with a ligase 310 to form concatenated fragments 312. That is, the blunting enzyme employed can exhibit exonuclease activity to digest (remove) the overhangs or polymerase activity to synthesize (fill-in) the missing complementary bases on the overhang. Some examples of blunting enzymes that can be used include, but are not limited to, DNA Polymerase I Klenow fragment, T4 DNA Polymerase, and Mung Bean Nuclease. In an exemplary embodiment, the blunting reagent mixture used to blunt the dsDNA concatenated fragments includes T4 DNA polymerase (which has 3′→5′ exonuclease activity and 5′→3′ polymerase activity) and T4 Polynucleotide Kinase (which aids in phosphorylation of 5′ ends of blunt ended DNA, necessary for subsequent ligation reaction).

After blunting ending 306 the 5′ and 3′ ends of the genomic fragments 302, a DNA ligase can be introduced to ligate the genomic fragments 302 to the genomic linker segments 308. During ligation 310, the DNA ligase seals the 5′ and 3′ polynucleotide ends via nucleotidyl transfer steps involving ligase-adenylate and DNA-adenylate intermediates. DNA ligases fall into two general categories: ATP-dependent DNA ligases (EC 6.5.1.1), and NAD (+) dependent DNA ligases (EC 6.5.1.2). NAD (+) dependent DNA ligases are found only in bacteria (and some viruses) while ATP-dependent DNA ligases are ubiquitous.

The ATP-dependent DNA ligases can be divided into four classes: DNA ligase I, II, III, and IV. DNA ligase I links Okazaki fragments to form a continuous strand of DNA; DNA ligase II is an alternatively spliced form of DNA ligase III, found only in non-dividing cells; DNA ligase III is involved in base excision repair; and DNA ligase IV is involved in the repair of DNA double-strand breaks by non-homologous end joining (NHEJ). Amongst all ligases, there are two types of prokaryotic and one type of eukaryotic ligases that are particularly well suited for facilitating the blunt ended double stranded DNA ligation: Prokaryotic DNA ligases (T3 and T4) and Eukaryotic DNA ligase (Ligase 1).

In some embodiments, T4 DNA ligase is used in the blunt end ligation process 310 for this protocol. Bacteriophage T4 DNA ligase is a single polypeptide with a M.W of about 68,000 Daltons requiring ATP as energy source. The maximal activity pH range is between about 7.5 to about 8.0. The presence of Mg++ ion is preferred and the optimal concentration is about 10 mM. T4 DNA ligase has the unique ability to join sticky and blunt ended fragments. T4 DNA ligase catalyzes phosphodiester bond formation between juxtaposed 5′ and 3′ termini in the genomic fragments 302 and genomic linker segments 308 in three steps: 1) enzyme-adenylylate formation by reaction with ATP; 2) adenylyl transfer to a 5-phosphorylated polynucleotide to generate adenylylated DNA; and 3) phosphodiester bond formation with release of AMP. In an exemplary embodiment, the ligation reaction can be carried out using 1 unit of T4 DNA ligase in a buffer consisting of 50 mM Tris HCl, 10 mM MgCl₂, 1 mM ATP and 10 mM DTT at a pH of about 7.5 and at a temperature of about 23° C. The reaction mixture containing the T4 ligase, blunt ended DNA and the linker segments can be incubated for 16-18 hours, without agitation. The concentration of the linker segment can range from about 1 pg to about 1 ng.

A concatenated fragment 312 forms once a genomic fragment 302 is ligated to a genomic linker segment 308. In some embodiments, the concatenated fragment 312 includes a least one genomic fragment 302 that is ligated to at least one genomic linker segment 308. In other embodiments, the concatenated fragment 312 includes two or more genomic fragments 302 and at least one genomic linker segment 308, whereby the at least one genomic fragment 302 is ligated to each end of the genomic linker segment 308. It should be appreciated, however, that a concatenated fragment 312 can have essentially any combination of genomic fragments 312 and genomic linker segments 308 as long as the combination is suitable for the purposes of sequencing and subsequent genomic feature analysis

After the formation of the concatenated fragments 312, they are amplified using WGA amplification technique 313 (such as PicoPlex, MDA, MALBAC, DOPlify etc.) and subsequently sequenced using a NGS (or equivalent) genomic sequencing system 316.

### Computer-Implemented System

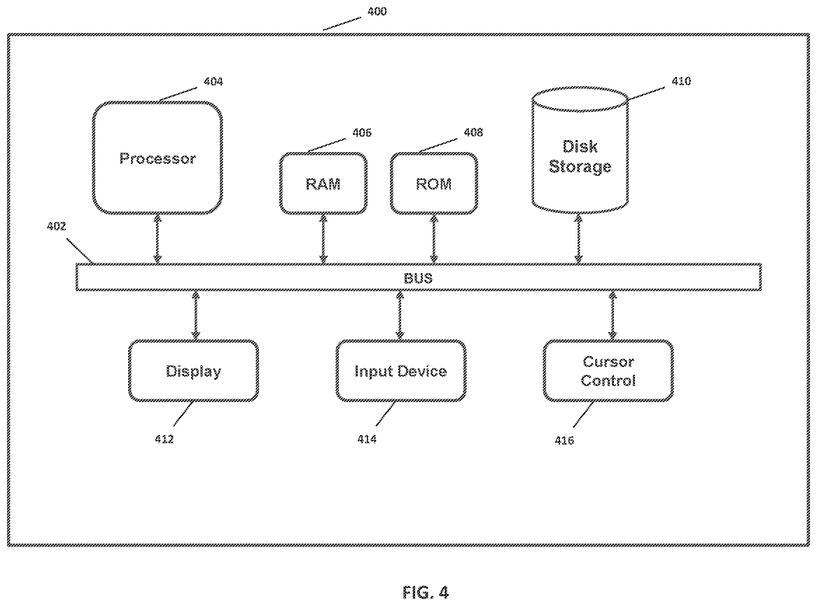

FIG. 4 is a block diagram that illustrates a computer system 400, upon which embodiments of the present teachings may be implemented. In various embodiments of the present teachings, computer system 400 can include a bus 402 or other communication mechanism for communicating information, and a processor 404 coupled with bus 402 for processing information. In various embodiments, computer system 400 can also include a memory, which can be a random access memory (RAM) 406 or other dynamic storage device, coupled to bus 402 for determining instructions to be executed by processor 404. Memory also can be used for storing temporary variables or other intermediate information during execution of instructions to be executed by processor 404. In various embodiments, computer system 400 can further include a read only memory (ROM) 408 or other static storage device coupled to bus 402 for storing static information and instructions for processor 404. A storage device 410, such as a magnetic disk or optical disk, can be provided and coupled to bus 402 for storing information and instructions.

In various embodiments, computer system 400 can be coupled via bus 402 to a display 412, such as a cathode ray tube (CRT) or liquid crystal display (LCD), for displaying information to a computer user. An input device 414, including alphanumeric and other keys, can be coupled to bus 402 for communicating information and command selections to processor 404. Another type of user input device is a cursor control 416, such as a mouse, a trackball or cursor direction keys for communicating direction information and command selections to processor 404 and for controlling cursor movement on display 412. This input device 414 typically has two degrees of freedom in two axes, a first axis (i.e., x) and a second axis (i.e., y), that allows the device to specify positions in a plane. However, it should be understood that input devices 414 allowing for 3 dimensional (x, y and z) cursor movement are also contemplated herein.

Consistent with certain implementations of the present teachings, results can be provided by computer system 400 in response to processor 404 executing one or more sequences of one or more instructions contained in memory 406. Such instructions can be read into memory 406 from another computer-readable medium or computer-readable storage medium, such as storage device 410. Execution of the sequences of instructions contained in memory 406 can cause processor 404 to perform the processes described herein. Alternatively hard-wired circuitry can be used in place of or in combination with software instructions to implement the present teachings. Thus implementations of the present teachings are not limited to any specific combination of hardware circuitry and software.

The term “computer-readable medium” (e.g., data store, data storage, etc.) or “computer-readable storage medium” as used herein refers to any media that participates in providing instructions to processor 404 for execution. Such a medium can take many forms, including but not limited to, non-volatile media, volatile media, and transmission media. Examples of non-volatile media can include, but are not limited to, optical, solid state, magnetic disks, such as storage device 410. Examples of volatile media can include, but are not limited to, dynamic memory, such as memory 406. Examples of transmission media can include, but are not limited to, coaxial cables, copper wire, and fiber optics, including the wires that comprise bus 402.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 404 of computer system 400 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc.

It should be appreciated that the methodologies described herein flow charts, diagrams and accompanying disclosure can be implemented using computer system 400 as a standalone device or on a distributed network of shared computer processing resources such as a cloud computing network.

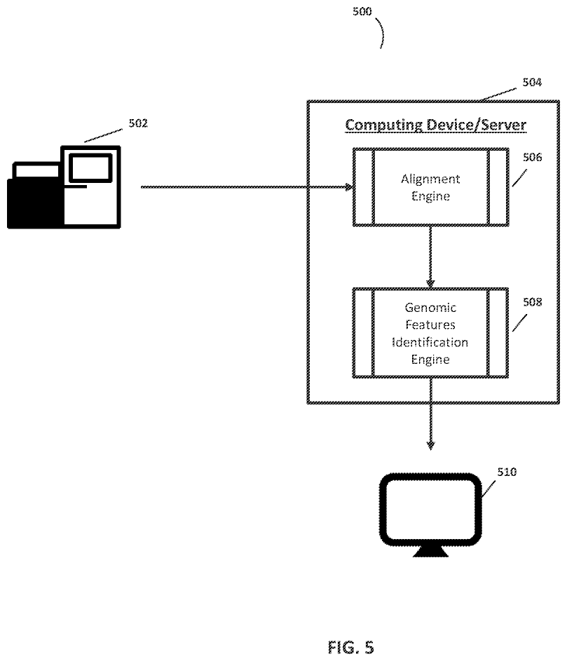

FIG. 5 is a schematic diagram of a system for non-invasive preimplantation genetic screening of embryos 500, in accordance with various embodiments. As depicted herein, the system 500 includes a genomic sequencing system 502, a computing device 504 and a display/client terminal 510.

In various embodiments, the computing device 504 can be communicatively connected to the genomic sequencing system 502 via a network connection that can be either a “hardwired” physical network connection (e.g., Internet, LAN, WAN, VPN, etc.) or a wireless network connection (e.g., Wi-Fi, WLAN, etc.). In various embodiments, the computing device 504 can be a workstation, mainframe computer, distributed computing node (part of a “cloud computing” or distributed networking system), personal computer, mobile device, etc. In various embodiments, the genomic sequencing system 504 can be a nucleic acid sequencer (e.g., NGS, Capillary Electrophoresis system, etc.), real-time/digital/quantitative PCR instrument, microarray scanner, etc. It should be understood, however, that the genomic sequencing system 504 can essentially be any type of instrument that can generate nucleic acid sequence data from samples containing genomic fragments.

It will be appreciated by one skilled in the art that various embodiments of genomic sequencing system 502 can be used to practice variety of sequencing methods including ligation-based methods, sequencing by synthesis, single molecule methods, nanopore sequencing, and other sequencing techniques. Ligation sequencing can include single ligation techniques, or change ligation techniques where multiple ligation are performed in sequence on a single primary nucleic acid sequence strand. Sequencing by synthesis can include the incorporation of dye labeled nucleotides, chain termination, ion/proton sequencing, pyrophosphate sequencing, or the like. Single molecule techniques can include continuous sequencing, where the identity of the nuclear type is determined during incorporation without the need to pause or delay the sequencing reaction, or staggered sequence, where the sequencing reactions is paused to determine the identity of the incorporated nucleotide.

In various embodiments, the genomic sequencing system 502 can determine the sequence of a nucleic acid, such as a polynucleotide or an oligonucleotide. The nucleic acid can include DNA or RNA, and can be single stranded, such as ssDNA and RNA, or double stranded, such as dsDNA or a RNA/cDNA pair. In various embodiments, the nucleic acid can include or be derived from a fragment library, a mate pair library, a chromatin immuno-precipitation (ChIP) fragment, or the like. In particular embodiments, the genomic sequencing instrument 502 can obtain the sequence information from a single nucleic acid molecule or from a group of substantially identical nucleic acid molecules.

In various embodiments, the genomic sequencing system 502 can output nucleic acid sequencing read data (genomic sequence information) in a variety of different output data file types/formats, including, but not limited to: *.fasta, *.csfasta, *.xsq, *seq.txt, *qseq.txt, *.fastq, *.sff, *prb.txt, *.sms, *srs and/or *.qv.

The analytics computing device 504 can be configured to host a sequence read alignment engine 506 and a genomic features identification engine 508. The read alignment engine 506 can be configure to receive genomic fragment sequence information generated by the genomic sequence system 502 and align (map) the genomic fragment sequences to a reference genome. Examples of publically available sequence alignment software that can be used to align the fragment sequences include BLAT, BLAST, Bowtie, BWA, drFAST LAST, MOSAIK, NEXTGENMAP, etc. Once the fragment sequences have been aligned, the genomic features identification engine 508 can be configured to identify genomic features on the aligned sequences. That is, the genomic features identification engine 508 can be communicatively connected (e.g., a network connection to the analytics computing device 504, a serial bus connection to database storage that is local to the analytics computing device 504, a peripheral device connection to a peripheral storage device connected to the analytics computing device 504, etc.) to various public (e.g., the RefGene Database (UCSC), the Alternative Splicing Database (EBI), the dbSNP database (NCBI), the Genomic Structural Variation database (NCBI), the GENCODE database (UCSC), the PolyPhen database (Harvard), the SIFT database (NCBI), the 3000 Genomes Project database, the Database of Genomic Variants database (EBI), the Biomart database (EBI), Gene Ontology database (public), the BioCyc/HumanCyc database, the KEGG pathway database, the Reactome database, the Pathway Interaction Database (NIH), the Biocarta database, PANTHER database, etc.) and private databases to identify the genomic features in the aligned sequences.

In some embodiments, the genomic features can be genomic variants such as insertions/deletions (INDEL), copy number variations (CNV), single nucleotide polymorphisms (SNP), duplications, inversions, translocations, etc. In other embodiments, the genomic features can be genomic regions that have some annotated function such as a gene, protein coding sequence, mRNA, tRNA, rRNA, repeat sequence, inverted repeat, miRNA, siRNA, etc. In still other embodiments, the genomic features can be epigenetic changes on the genome (e.g., methylation, acetylation, ubiquitylation, phosphorylation, sumoylation, ribosylation, citrullination, etc.) that can affect gene expression and activity.

It should be appreciated that the functionalities of the read alignment engine 506 and genomic features identification engine 508 can be implemented as hardware, firmware, software, or any combination thereof. Furthermore, the various engines depicted in FIG. 5 can be combined or collapsed into a single engine, component or module, depending on the requirements of the particular application or system architecture. Moreover, in various embodiments, the read alignment engine 506 and genomic features identification engine 508 can comprise additional engines or components as needed by the particular application or system architecture.

After the genomic features have been identified, the results can be displayed on a display or client terminal 510 that is communicatively connected to the computing device 504. In various embodiments, client terminal 510 can be a thin client computing device. In various embodiments, client terminal 510 can be a personal computing device having a web browser (e.g., INTERNET EXPLORER™, FIREFOX™ SAFARI™ etc) that can be used to control the operation of the sequence alignment engine 506 and/or genomic features identification engine 508. That is, the client terminal 510 can access the sequence alignment engine 506 using a browser to control the operation of the sequence alignment engine 506. For example, the sequence alignment criteria or logic can be modified depending on the requirements of the particular application. Similarly, client terminal 510 can access the genomic features identification engine 508 using a browser to control the database sources (e.g., the RefGene Database (UCSC), the Alternative Splicing Database (EBI), the dbSNP database (NCBI), the Genomic Structural Variation database (NCBI), the GENCODE database (UCSC), the PolyPhen database (Harvard), the SIFT database (NCBI), the 3000 Genomes Project database, the Database of Genomic Variants database (EBI), the Biomart database (EBI), Gene Ontology database (public), the BioCyc/HumanCyc database, the KEGG pathway database, the Reactome database, the Pathway Interaction Database (NTH), the Biocarta database, PANTHER database, etc.) used to identify the genomic features in the aligned sequences or the modify the summary reports generated.

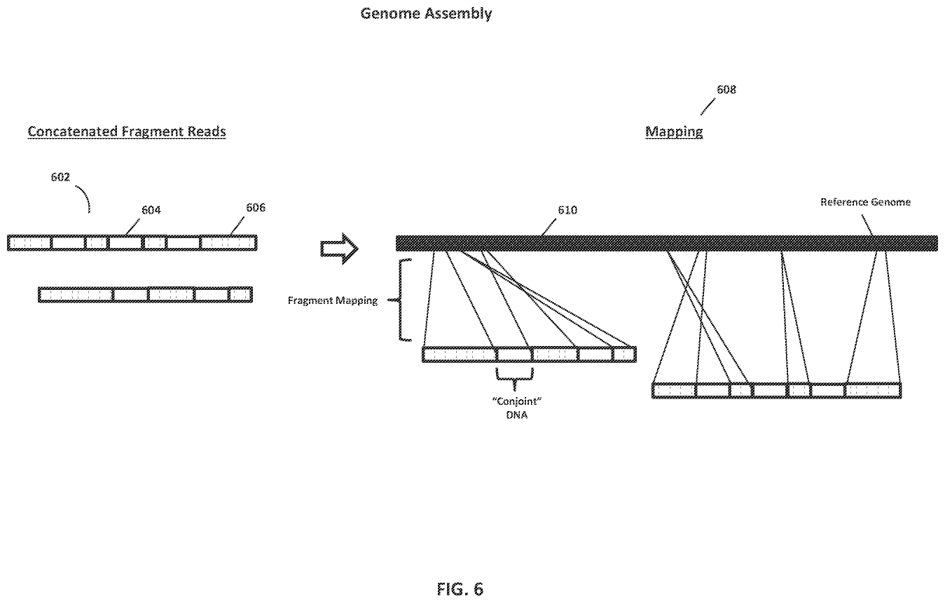

FIG. 6 is a depiction of how concatenated fragment reads are mapped to a reference genome, in accordance with various embodiments. As discussed previously, concatenated fragments are comprised of both genomic fragments that the candidate embryo has secreted or shed (in the media that it was incubated in) and artificially created double-stranded “conjoint” oligonucleotide segments (i.e., genomic linker segments) of a known length and nucleotide (base) sequence. Therefore, as depicted herein FIG. 6, the concatenated fragment reads 602 are comprised of sequence reads of both the artificially synthesized genomic linker segments 604 and the genomic fragments 606 obtained from the embryo test media.

The concatenated fragment reads 602 are aligned (mapped) 608 to a reference genome 610 using any number of publically available sequence alignment tools including, but not limited to: BLAT, BLAST, BWA, Bowtie, drFAST LAST, MOSAIK, NEXTGENMAP, etc. In some embodiments, the parameters of the sequence alignment tool are modified to accommodate short fragment sequence read alignments. In some embodiments, the short genomic fragment reads have a length of between about 30 base pairs (bps) and about 800 bps. In other embodiments, the short genomic fragment reads have a length of between bout 150 bps to about 400 bps. In still other embodiments, the short genomic fragment reads have a length of less than about 1000 bps.

In some embodiments, the genomic linker segments sequence reads are between about 30 to 1000 bps in length. In other embodiments, the genomic linker segment sequence reads are between about 30 bps and about 500 bps in length. In still other embodiments, the genomic linker segment sequence reads are between about 50 bps to about 150 bps. In some embodiments, the genomic linker segment sequence reads are homopolymer sequences. In other embodiments, the genomic linker segment sequence reads are heteropolymer oligonucleotide sequences.

In some embodiments, since the genomic linker segment sequence reads are not naturally occurring they are algorithmically filtered out during the alignment of the concatenated fragment reads to the reference genome. That is, the alignment tool subtracts out the known sequences associated with the genomic linker segments and only aligns the sequences associated with the genomic fragments portion of the concatenated fragment reads to the reference genome.

In some embodiments, the alignment tool selects the best alignment for each genomic fragment sequence read by determining the longest matching alignment position on the reference genome for each genomic fragment sequence read. That is, the alignment location where the longest consecutive sequence of bases on the genomic fragment sequence read matches to the reference genome. In other embodiments, the alignment tool selects the best alignment for each genomic fragment sequence read by determining the position on the reference genome where the most number of bases from the genomic fragment sequence reads match, regardless of whether they are consecutive or not.

In some embodiments, genomic fragment sequence reads that align equally well to multiple locations on the reference genome are automatically discarded and not used in the identification of genomic features (e.g., SNPs, CNVs, Indels, etc.).

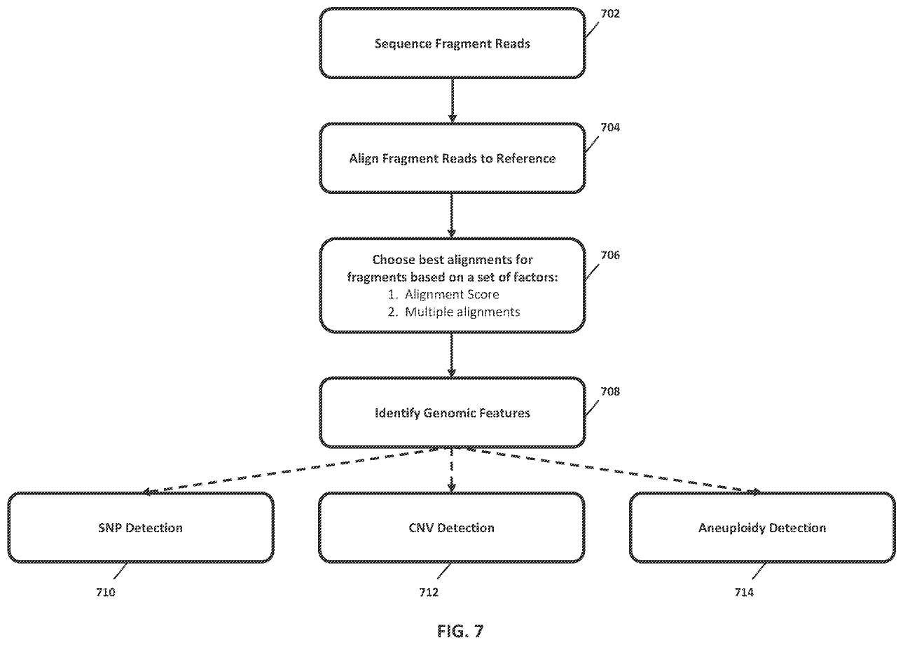

FIG. 7 is an exemplary flowchart showing a method for aligning concatenated genomic fragment sequence reads to identify various types of genomic features, in accordance with various embodiments. As depicted herein, the concatenated genomic fragment sequence reads 702 are first aligned to a reference genome 704. The alignments are made using any number of publically available sequence alignment tools including, but not limited to: BLAT, BLAST, BWA, Bowtie, drFAST LAST, MOSAIK, NEXTGENMAP, etc. As discussed above, the concatenated genomic fragment reads are sequence reads of both the artificially synthesized genomic linker segments and the genomic fragments obtained from the test sample (e.g., tissue, embryo, etc.).

In some embodiments, since the genomic linker segments are not naturally occurring (in the human genome) they are algorithmically filtered out during the alignment of the concatenated fragment reads to the reference genome. That is, the alignment tool subtracts out the known sequences associated with the genomic linker segments and only aligns the sequences associated with the genomic fragments portion of the concatenated fragment reads to the reference genome.

The alignment tool selects the best alignment for each genomic fragment sequence read based on a set of parameters or factors 706, including, but not limited to, alignment score and whether there are multiple alignments for the genomic fragment reads. In some embodiments, the alignment score for a genomic fragment read alignment can be calculated (using Equation 1) as a function of a match criteria (e.g., a number of consecutive bases of the genomic fragment sequence read that matches to the reference genome, the absolute number of bases from the genomic fragment sequence read that matches to the reference genome, the percent sequence identity between the sequence and its match in the genome, etc.), a mismatch criteria and gap penalties. Within the construct of Equation 1, mismatches and gaps in alignment are penalized from the overall alignment score.

Alignment Score=f(match criteria)−f(mismatch criteria)−f(Gap Penalties) Equation 1:

In some embodiments, genomic fragment sequence reads that align equally well (e.g., have the same alignment score, etc.) to multiple locations on the reference genome are automatically discarded and not used in the identification of genomic features.

After the genomic fragment sequence reads 702 are aligned to the reference genome, various analytics tools or callers can be used to identify genomic features on the aligned sequences 708. In various embodiments, these tools or callers can be configured to access various public (e.g., the RefGene Database (UCSC), the Alternative Splicing Database (EBI), the dbSNP database (NCBI), the Genomic Structural Variation database (NCBI), the GENCODE database (UCSC), the PolyPhen database (Harvard), the SIFT database (NCBI), the 3000 Genomes Project database, the Database of Genomic Variants database (EBI), the Biomart database (EBI), Gene Ontology database (public), the BioCyc/HumanCyc database, the KEGG pathway database, the Reactome database, the Pathway Interaction Database (NTH), the Biocarta database, PANTHER database, etc.) and/or private databases to identify the genomic features.

In some embodiments, the genomic features can be genomic variants such as insertions/deletions (INDEL), copy number variations (CNV), single nucleotide polymorphisms (SNP), duplications, inversions, translocations, etc. In other embodiments, the genomic features can be genomic regions that have some annotated function such as a gene, protein coding sequence, mRNA, tRNA, rRNA, repeat sequence, inverted repeat, miRNA, siRNA, etc. In still other embodiments, the genomic features can be epigenetic changes on the genome (e.g., methylation, acetylation, ubiquitylation, phosphorylation, sumoylation, ribosylation, citrullination, etc.) that can affect gene expression and activity.

In various embodiments, SNPs can be called via local de-novo assembly of haplotypes 710. In various embodiments, aneuploiday can be called using an aneuploidy caller 714. In various embodiments, Copy Number Variants (CNVs) can be identified using a modified CNV caller 712. The modified CNV caller can be configured to differentiate between biological and technical variation by normalization to a normal sample. Technical variations can occur due to bias in technology, for example, some regions in the genome can have more or less reads when sequenced due to high GC content bias (i.e., the proportion of G and C bases in a region and the count of fragments mapped to it), amplification bias, linker ligation etc. so they are not real CNV deletions or duplications; but instead, are merely experimental artifacts. On the other hand, biological variations are due to actual CNV deletions/duplications in the genome. For example, when the genome region (i.e., chromosomal position) of the sample (e.g., tissue, embryo, etc.) being tested has a CNV deletion it will have less reads in that region and when the genome has a CNV duplication it means that it has more reads in that region. In various embodiments, in order to remove bias from technical variations and be able to differentiate between “real” biological variations from “fake” technical variations a circular binary segmentation (CBS) based algorithm is applied and spline normalization is performed using an Interpolated Univariate smoothing model.

That is, normalizations are performed to compare regions of one sample to all other samples that have been previously tested. The logic being if there are technical variations they will affect all the samples within a sample test batch (i.e., the samples that are run through the amplification and sequencing workflow steps together) and not just one sample within a batch of samples. So if a sample shows a drop in the quantity of reads in a region which is also seen in other samples of the same sample batch then it is safe to conclude that it was a technical variation. However, if the drop is only seen in one sample in a sample batch and in no other sample in the same sample batch then it is highly likely to be a biological variation. This comparison can be done only when all samples are normalized to the same scale. To do this, gene regions of interest are typically split into many small intervals of approximately 100 bps and the average depths (i.e., quantity of aligned reads) of the samples are calculated for each region. Even if individual interval shows variation, the Spline normalization performed smooths over the region, so that it removes smaller errors so that only significant variations in each region will be detectable. CNVs can then be identified by measuring significance using techniques such as Principal Component Analysis (PCA).

In various embodiments, the CBS algorithm is configured to identify the start and end positions for CNVs in a sample. That is, the CBS algorithm performs multiple passes through a sample whereby on the first pass the algorithm searches the entire sample, compiling a list of (start, end) position tuples in which statistically significant changes in read depth appear to have occurred. Among these tuples, the tuple containing the most dramatic change is identified as a CNV, and then the algorithm is reapplied recursively to the two pieces of the sample on either side of this tuple. The algorithm terminates when no statistically significant changes in read depth occur in any of the portions of the sample currently under evaluation.

Put another way, for every small interval, the CBS algorithm compares the intervals before and after it and if they both show the same drop/increase it moves to the next interval. At the boundary of the variation, one side will have the signal while the other won't, which helps define the boundaries.

In various embodiments, during the Spline normalization of the genome regions (i.e., chromosomal positions) in the genome of a sample being tested for CNV, a quantiling function is used to partition by depth the reads for a particular sample to ascertain what constitutes a low, average and deep read depth for each genome region. The same procedure is then repeated for the median read depth at each genome region in the genome across all samples in the batch.

The breakpoints which partition these read depths by low, average, deep, etc. for a particular sample are plotted on the x-axis, and the breakpoints which partition the read depths for the median across samples is plotted on the y-axis. These (x, y) values are then interpolated with a curve.

Next, for a particular sample, the read depth for a particular region in said sample is evaluated against the curve, by looking at the height on the curve corresponding to its region on the x-axis. By doing this, samples which have, for example, a large percentage of low coverage regions when compared to the median across samples will be modified in such a way that the upper portion of their low coverage regions will be re-interpreted as being of average coverage. Next, if a sample shows a drop in reads in a region which is also seen in other samples then it can be classified as a technical variation, however if the drop is only seen in one sample and in no other sample in the batch then it can be classified as a biological variation. This is accounted for by dividing a sample's read depth at a particular region by the median read depth at that same region across all samples in a batch.

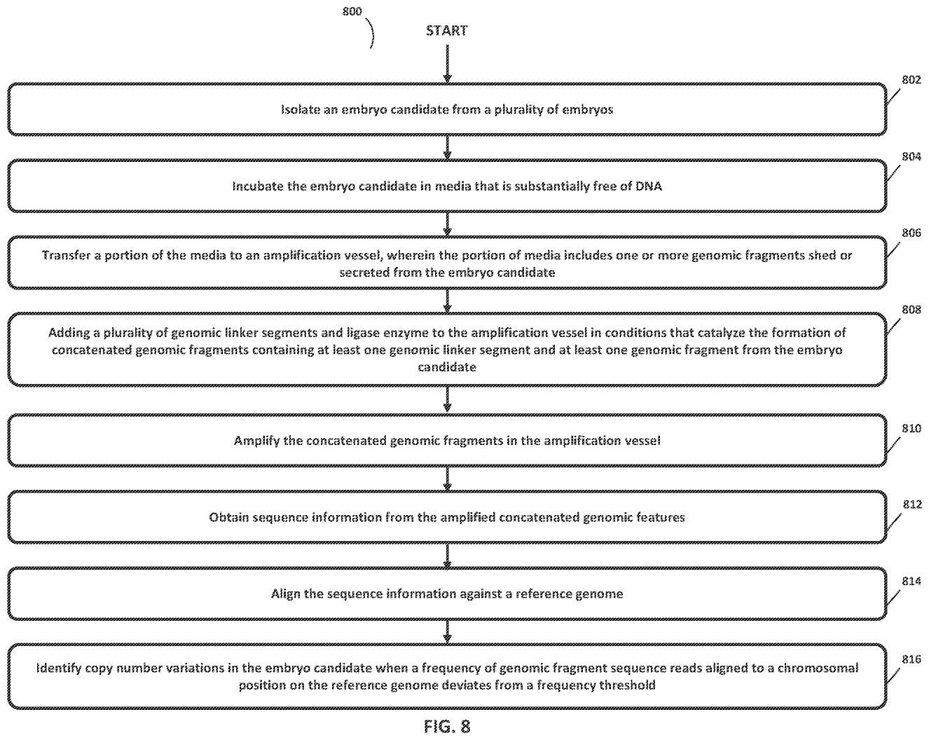

FIG. 8 is a flowchart showing a method for determining copy number variation in an embryo candidate, in accordance with various embodiments. As depicted herein, method 800 details an exemplary workflow for identifying copy number variations in an embryo candidate. In step 802, an embryo candidate is isolated from a plurality of fertilized embryos and placed into a container. For example, the embryo candidate can be isolated from a plurality of fertilized embryos each of which can be a candidate for IVF implantation. In some embodiments, the embryo candidate is in the blastocyst stage of embryongenesis. In some embodiments, the embryo candidate is a human embryo.

Typically, isolation step 802 is performed using conventional sterile techniques or in a sterile hood to ensure that the isolated embryo candidate is not contaminated with genomic matter that may lead to erroneous test results.

In step 804, the embryo candidate is incubated in media that is substantially free of DNA. Typically, the embryo is incubated for as long of a period of time as is required (while still keeping the embryo candidate viable for IVF implantation) for a sufficient quantity of DNA fragments (i.e., genomic fragments) to be secreted or shed from the embryo candidate to the DNA free media for a copy number variation analysis to be performed using method 800. In some embodiments, the embryo can be incubated in the culture media for a minimum of about 18 hrs. In other embodiments, the embryo can be incubated in the culture media for between about 18 hours and about 144 hours. An example of DNA free media that can be utilized in this workflow is ORIGIO SEQUENTIAL BLAST™ culture media of The Cooper Companies. In various embodiments, the media can be substantially free of oligonucletides and not just DNA to ensure the lowest possible chance of erroneous analysis results or artifact formation during amplification.

In step 806, a portion of the media is transferred to an amplification vessel, wherein the portion of media includes one or more genomic fragments (i.e., DNA fragment) shed or secreted from the embryo candidate. Examples of an amplification vessel that can be used include, but are not limited to, a test tube, pipette tube, petri dish, or a well/partition within a multi-partition/well plate.

In step 808, a plurality of linker segments and ligase enzyme is added to the amplification vessel in conditions that catalyze the formation of concatenated genomic fragments containing at least one genomic linker segment and at least one genomic fragment (from the embryo candidate). Typically, the genomic fragments obtained from the media are considered “short” genomic fragments. In some embodiments, the short genomic fragments have lengths of between about 30 base pairs (bps) and about 800 bps. In other embodiments, the short genomic fragments have a length of between about 150 bps to about 400 bps. In still other embodiments, the short genomic fragments have a length of less than about 1000 bps.

The genomic linker segments are essentially artificially created double-stranded “conjoint” oligonucleotide segments of a known length and nucleotide sequence. In some embodiments, the genomic linker segments are between about 30 to 1000 bps in length. In other embodiments, the genomic linker segments are between about 30 bps and about 500 bps in length. In still other embodiments, the genomic linker segments are between about 50 bps to about 150 bps. In some embodiments, the genomic linker segments are homopolymer oligonucleotide segments. In other embodiments, the genomic linker segments are heteropolymer oligonucleotide segments. In some embodiments, the genomic linker segments are blunt ended double-stranded oligonucleotide segments. In some embodiments, the genomic fragments are enzymatically blunt ended prior to being ligated to the genomic linker segments using methods that were previously disclosed above.

Various types of prokaryotic and eukaryotic enzymes (i.e., ligases) can be used to ligate the genomic fragments to the genomic linker segments to form the concatenated genomic fragments. Some examples of ligases that can be used here include, but are not limited to, T3, T4, T7, or Ligase 1.

In step 810, the concatenated genomic fragments are amplified in the amplification vessel. In various embodiments, the concatenated genomic fragments are amplified on a thermal cycler (or similar device) using WGA techniques such as MDA, MALBAC, etc.

Because the concatenated fragments are significantly longer than the original genomic fragments isolated from the incubation media, amplification errors are significantly reduced (when compared to amplifying the genomic fragments individually).

In step 812, sequence information from the amplified concatenated genomic fragments is obtained from sequencing the concatenated fragments on a NGS or equivalent genomic sequencing system. In some embodiments, the sequence information includes both genomic fragment sequence reads (obtained from genomic fragments isolated from the embryo candidate) and genomic linker segment sequence reads (which were artificially created and ligated to the genomic fragments prior to amplification in step 810).

In step 814, the sequence information is aligned against a reference genome using a publically available or proprietary sequence alignment tool. Examples of publically available sequence alignment tools that can be used to align the fragment sequences include, but are not limited to, BLAT, BLAST, BWA, Bowtie, drFAST LAST, MOSAIK, NEXTGENMAP, etc. In some embodiments, since the genomic linker segments are not naturally occurring their corresponding sequence reads are algorithmically filtered out during the alignment of the sequence information to the reference genome. That is, the alignment tool subtracts out the known sequences associated with the genomic linker segments and only aligns the sequences associated with the genomic fragments portion of the concatenated fragment reads to the reference genome.

In some embodiments, the alignment tool selects the best alignment for each genomic fragment sequence read by determining the longest matching alignment position on the reference genome for each genomic fragment sequence read. That is, the alignment location where the longest consecutive sequence of bases on the genomic fragment sequence read matches to the reference genome. In other embodiments, the alignment tool selects the best alignment for each genomic fragment sequence read by determining the position on the reference genome where the most number of bases from the genomic fragment sequence reads match, regardless of whether they are consecutive or not. In some embodiments, genomic fragment sequence reads that align equally well to multiple locations on the reference genome are automatically discarded and not used.

In step 816, copy number variations in the embryo candidate's genome are identified when a frequency of genomic fragment sequence reads aligned to a chromosomal position on the reference genome deviates from a frequency threshold. In various embodiments, a deviance occurs when the frequency of genomic fragment sequences aligned to a chromosomal position is below the frequency threshold (i.e., fragment alignment frequency in a normal genome). That is, when the chromosomal position of the sample (e.g., tissue, embryo, etc.) being tested has a CNV deletion it will have less reads (i.e. frequency of reads aligned) in that region than in a normal genome. In various embodiments, a deviance occurs when the frequency of genomic fragment sequences aligned to a chromosomal position is above the frequency threshold. That is, when the chromosomal position has CNV duplication it means that it has more reads in that region than in a normal genome.

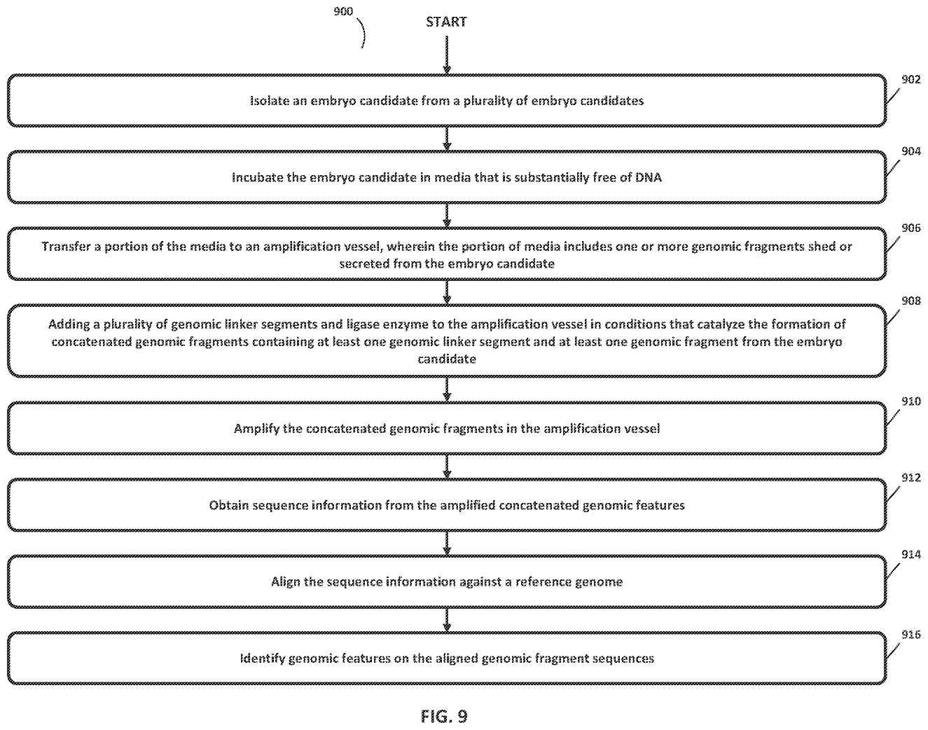

FIG. 9 is a flowchart showing a method of identifying genomic features in an embryo candidate, in accordance with various embodiments. As depicted herein, method 900 details an exemplary workflow for identifying genomic features in an embryo candidate. In step 902, an embryo candidate is isolated from a plurality of embryo candidates. For example, the embryo candidate can be isolated from a plurality of fertilized embryos each of which can be a candidate for IVF implantation. In some embodiments, the embryo candidate is in the blastocyst stage of embryongenesis. In some embodiments, the embryo candidate is a human embryo.

In step 904, the embryo candidate is incubated in media that is substantially free of DNA. Typically, the embryo is incubated for as long of a period of time as is required (while still keeping the embryo candidate viable for IVF implantation) for a sufficient quantity of DNA fragments (i.e., genomic fragments) to be secreted or shed from the embryo candidate to the DNA free media for a copy number variation analysis to be performed using method 900. An example of DNA free media that can be utilized in this workflow is ORIGIO SEQUENTIAL BLAST™ culture media of The Cooper Companies. In various embodiments, the media can be substantially free of oligonucleotides and not just DNA to ensure the lowest possible chance of erroneous analysis results or artifact formation during amplification.

In step 906, a portion of the media is transferred to an amplification vessel, wherein the portion of media includes one or more genomic fragments (i.e., DNA fragment) shed or secreted from the embryo candidate. Examples of an amplification vessel that can be used include, but are not limited to, a test tube, pipette tube, petri dish, or a well/partition within a multi-partition/well plate.

In step 908, a plurality of linker segments and ligase enzyme is added to the amplification vessel in conditions that catalyze the formation of concatenated genomic fragments containing at least one genomic linker segment and at least one genomic fragment from the embryo candidate. Typically, the genomic fragments isolated from the media are considered “short” genomic fragments. In some embodiments, the short genomic fragments have lengths of between about 30 base pairs (bps) and about 800 bps. In other embodiments, the short genomic fragments have lengths of between bout 150 bps to about 400 bps. In still other embodiments, the short genomic fragments have lengths of less than about 1000 bps.

The genomic linker segments are essentially artificially created double-stranded “conjoint” oligonucleotide segments of a known length and nucleotide sequence. In some embodiments, the genomic linker segments are between about 30 to about 1000 bps in length. In other embodiments, the genomic linker segments are between about 30 bps and about 500 bps in length. In still other embodiments, the genomic linker segments are between about 50 bps to about 150 bps. In some embodiments, the genomic linker segments are homopolymer oligonucleotide segments. In other embodiments, the genomic linker segments are heteropolymer oligonucleotide segments. In some embodiments, the genomic linker segments are blunt ended double-stranded oligonucleotide segments. In some embodiments, the genomic fragments are enzymatically blunt ended prior to being ligated to the genomic linker segments using methods that were previously disclosed above.

Various types of prokaryotic and eukaryotic enzymes (i.e., ligases) can be used to ligate the genomic fragments to the genomic linker segments to form the concatenated genomic fragments. Some examples of ligases that can be used here include, but are not limited to, T3, T4, T7, or Ligase 1.

In step 910, the concatenated genomic fragments are amplified in the amplification vessel. In various embodiments, the concatenated genomic fragments are amplified on a thermal cycler (or similar device) using WGA techniques such as MDA, MALBAC, etc.

In step 912, sequence information from the amplified concatenated genomic features are obtained from sequencing the concatenated fragments on a NGS or equivalent genomic sequencing system. In some embodiments, the sequence information includes both genomic fragment sequence reads (obtained from genomic fragments isolated from the embryo candidate) and genomic linker segment sequence reads (which were artificially created and ligated to the genomic fragments prior to amplification in step 910).

In step 914, the sequence information is aligned against a reference genome using a publically available or proprietary sequence alignment tool. Examples of publically available sequence alignment tools that can be used to align the fragment sequences include, but are not limited to, BLAT, BLAST, BWA, Bowtie, drFAST LAST, MOSAIK, NEXTGENMAP, etc. In some embodiments, since the genomic linker segments are not naturally occurring their corresponding sequence reads are algorithmically filtered out during the alignment of the sequence information to the reference genome. That is, the alignment tool subtracts out the known sequences associated with the genomic linker segments and only aligns the sequences associated with the genomic fragments portion of the concatenated fragment reads to the reference genome.

In some embodiments, the alignment tool selects the best alignment for each genomic fragment sequence read by determining the longest matching alignment position on the reference genome for each genomic fragment sequence read. That is, the alignment location where the longest consecutive sequence of bases on the genomic fragment sequence read matches to the reference genome. In other embodiments, the alignment tool selects the best alignment for each genomic fragment sequence read by determining the position on the reference genome where the most number of bases from the genomic fragment sequence reads match, regardless of whether they are consecutive or not. In some embodiments, genomic fragment sequence reads that align equally well to multiple locations on the reference genome are automatically discarded and not used.

In step 916, genomic features are identified on the aligned genomic fragment sequences using a various publically available or proprietary genomic features analytics tools or callers. In various embodiments, these tools or callers can be configured to access various public (e.g., the RefGene Database (UCSC), the Alternative Splicing Database (EBI), the dbSNP database (NCBI), the Genomic Structural Variation database (NCBI), the GENCODE database (UCSC), the PolyPhen database (Harvard), the SIFT database (NCBI), the 3000 Genomes Project database, the Database of Genomic Variants database (EBI), the Biomart database (EBI), Gene Ontology database (public), the BioCyc/HumanCyc database, the KEGG pathway database, the Reactome database, the Pathway Interaction Database (NIH), the Biocarta database, PANTHER database, etc.) and/or private databases to identify the genomic features.

In some embodiments, the genomic features can be genomic variants such as insertions/deletions (INDEL), copy number variations (CNV), single nucleotide polymorphisms (SNP), duplications, inversions, translocations, etc. In other embodiments, the genomic features can be genomic regions that have some annotated function such as a gene, protein coding sequence, mRNA, tRNA, rRNA, repeat sequence, inverted repeat, miRNA, siRNA, etc. In still other embodiments, the genomic features can be epigenetic changes on the genome (e.g., methylation, acetylation, ubiquitylation, phosphorylation, sumoylation, ribosylation, citrullination, etc.) that can affect gene expression and activity.

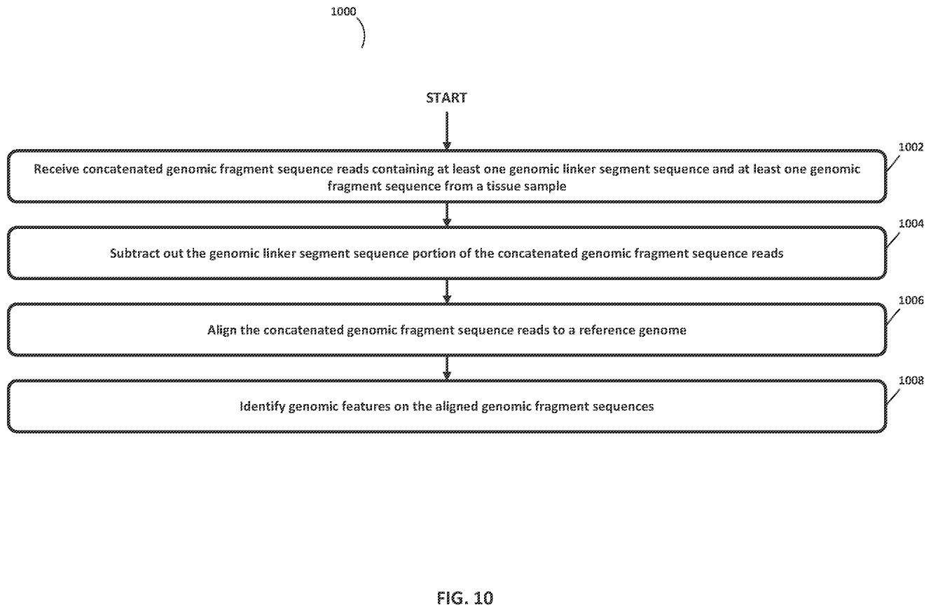

FIG. 10 is a flowchart showing a method for identifying genomic features from concatenated genomic fragment sequence reads, in accordance with various embodiments. As depicted herein, method 1000 details an exemplary workflow for identifying genomic features on genomic fragment sequence reads that were obtained from concatenated fragments (created by ligating artificial genomic linker segments to genomic fragments that were extracted from a tissue sample) that were amplified and later sequenced on a NGS or equivalent genomic sequencing system. In step 1002, concatenated genomic fragment reads containing at least one genomic linker segment sequence and at least one genomic fragment sequence from a tissue sample is received on a computing device/server programmed with instructions (software or hardware) to analyze genomic sequence information (sequence reads) generated by a genomic sequencing system configured to determine the base sequence information of genomic fragments.

The genomic linker segments are artificially created so their length and base sequence isn't known. In some embodiments, the genomic linker segment reads are between about 30 to about 1000 bps in length. In other embodiments, the genomic linker segment reads are between about 30 bps and about 500 bps in length. In still other embodiments, the genomic linker segment reads are between about 50 bps to about 150 bps. In some embodiments, the genomic linker segment reads are homopolymer sequences. In other embodiments, the genomic linker segment reads are heteropolymer sequences.

In step 1004, the genomic linker segment sequence portion of the concatenated genomic fragment sequence reads is subtracted out prior to the concatenated genomic fragment sequence reads being aligned to a reference genome in step 1006. That is, the known sequences associated with the genomic linker segments is subtracted out from the concatenated genomic fragment sequence reads first and then only the genomic fragments portion of the concatenated fragment reads are aligned to the reference genome.

In step 1008, genomic features are identified on the aligned genomic fragment sequences using various publically available or proprietary genomic features analytics tools or callers. In various embodiments, these tools or callers can be configured to access various public (e.g., the RefGene Database (UCSC), the Alternative Splicing Database (EBI), the dbSNP database (NCBI), the Genomic Structural Variation database (NCBI), the GENCODE database (UCSC), the PolyPhen database (Harvard), the SIFT database (NCBI), the 3000 Genomes Project database, the Database of Genomic Variants database (EBI), the Biomart database (EBI), Gene Ontology database (public), the BioCyc/HumanCyc database, the KEGG pathway database, the Reactome database, the Pathway Interaction Database (NIH), the Biocarta database, PANTHER database, etc.) and/or private databases to identify the genomic features.

In some embodiments, the genomic features can be genomic variants such as insertions/deletions (INDEL), copy number variations (CNV), single nucleotide polymorphisms (SNP), duplications, inversions, translocations, etc. In other embodiments, the genomic features can be genomic regions that have some annotated function such as a gene, protein coding sequence, mRNA, tRNA, rRNA, repeat sequence, inverted repeat, miRNA, siRNA, etc. In still other embodiments, the genomic features can be epigenetic changes on the genome (e.g., methylation, acetylation, ubiquitylation, phosphorylation, sumoylation, ribosylation, citrullination, etc.) that can affect gene expression and activity.

### Experimental Results

The following illustrative example is a representative embodiment of the software applications, systems, and methods described herein and are not meant to be limiting in any way.

As shown in the Table 1, 26 embryos were analyzed for chromosomal abnormalities (i.e., CNV) using both a conventional embryo trophectoderm biopsy method and the novel non-invasive sampling of embryo culture media methods disclosed above. The samples were either analyzed immediately or stored in temperature conditions ranging from −20° C. to −80° C.

TABLE 1

| Aneuploidy | Euploidy | Individual Chromosome |
|---|---|---|
| 100% (8/8) | 89% (16/18) | 99% (615/624) |

The DNA of each traditional embryo biopsy was analyzed using ILLUMINA's VERISEQ™ PGS workflow and analysis. IVF culture media for each respective embryo was subjected to noninvasive analysis via a novel amplification method, sequenced on an ILLUMINA NGS sequencer and their chromosome copy numbers were calculated using a custom bioinformatics pipeline. The results clearly show high concordance in the aneupoloidy (chromosomal abnormality) and euploidy (normal genetic makeup) calls between the industry accepted trophectoderm biopsy method and the non-invasive embryo culture media methods disclosed above. Moreover, there was a high concordance rate for each of the 24 chromosomes that were compared across each respective sample.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 400 of FIG. 4, whereby processor 404 would execute the analyses and determinations provided by these engines, subject to instructions provided by any one of, or a combination of, memory components 406/4008/410 and user input provided via input device 414.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Further, in describing various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments.

The embodiments described herein, can be practiced with other computer system configurations including hand-held devices, microprocessor systems, microprocessor-based or programmable consumer electronics, minicomputers, mainframe computers and the like. The embodiments can also be practiced in distributing computing environments where tasks are performed by remote processing devices that are linked through a network.

It should also be understood that the embodiments described herein can employ various computer-implemented operations involving data stored in computer systems. These operations are those requiring physical manipulation of physical quantities. Usually, though not necessarily, these quantities take the form of electrical or magnetic signals capable of being stored, transferred, combined, compared, and otherwise manipulated. Further, the manipulations performed are often referred to in terms, such as producing, identifying, determining, or comparing.

Any of the operations that form part of the embodiments described herein are useful machine operations. The embodiments, described herein, also relate to a device or an apparatus for performing these operations. The systems and methods described herein can be specially constructed for the required purposes or it may be a general purpose computer selectively activated or configured by a computer program stored in the computer. In particular, various general purpose machines may be used with computer programs written in accordance with the teachings herein, or it may be more convenient to construct a more specialized apparatus to perform the required operations.

Certain embodiments can also be embodied as computer readable code on a computer readable medium. The computer readable medium is any data storage device that can store data, which can thereafter be read by a computer system. Examples of the computer readable medium include hard drives, network attached storage (NAS), read-only memory, random-access memory, CD-ROMs, CD-Rs, CD-RWs, magnetic tapes, and other optical, FLASH memory and non-optical data storage devices. The computer readable medium can also be distributed over a network coupled computer systems so that the computer readable code is stored and executed in a distributed fashion.

### Recitation of Selected Embodiments

Embodiment 1. A method is provided for determining copy number variation in an embryo candidate for in vitro fertilization (IVF) implantation is disclosed. An embryo candidate is isolated from a plurality of embryos. The embryo candidate is incubated in media that is substantially free of DNA. A portion of the media is transferred to an amplification vessel, wherein the portion of media includes genomic fragments shed or secreted from the embryo candidate. A plurality of genomic linker segments and ligase enzyme is added to the amplification vessel in conditions that catalyze the formation of concatenated genomic fragments containing at least one genomic linker segment and at least one genomic fragment from the isolated embryo candidate. The concatenated genomic fragments are amplified in the amplification vessel. Sequence information is obtained from the amplified concatenated genomic fragments. The sequence information is aligned (mapped) against a reference genome. Copy number variations are identified in the embryo candidate when a frequency of genomic fragment sequence reads aligned to a chromosomal position on the reference genome deviates from a frequency threshold.

Embodiment 2. The method of Embodiment 1, further including: subtracting sequence information related to the genomic linker segment from the concatenated genomic fragment sequence prior to aligning the concatenated genomic fragment sequence to the reference genome.

Embodiment 3. The method of Embodiment 2, further including: normalizing the frequency of genomic fragment sequence reads aligned to each chromosomal position; and determining a frequency threshold for each chromosomal position.

Embodiment 4. The method of Embodiment 3, further including: applying a circular binary segmentation (CBS) analysis to determine whether the identified deviance from the frequency threshold identified is due to technical bias.

Embodiment 5. The method of Embodiment 3, wherein the normalization is performed using a Spline normalization method.

Embodiment 6. The method of Embodiment 1, further including: blunting the genomic fragment ends using a modified polymerase prior to ligating them to the genomic linker segments.

Embodiment 7. The method of Embodiment 6, wherein the modified polymerase is a Klenow T4 DNA polymerase.

Embodiment 8. The method of Embodiment 1, wherein the ligase enzyme is one of a T3, T4 or T7 prokaryotic DNA ligase.

Embodiment 9. The method of Embodiment 1, wherein the embryo candidate is a human embryo.

Embodiment 10. The method of Embodiment 1, wherein the embryo candidate is a blastocyst.

Embodiment 11. The method of Embodiment 1, wherein the frequency threshold is a frequency of genomic fragment reads that map to a normal chromosome.

Embodiment 12. A method is provided for identifying genomic features in an embryo candidate is disclosed. An embryo candidate is isolated from a plurality of embryo candidates. The embryo candidate is incubated in media that is substantially free of DNA. A portion of the media is transferred to an amplification vessel, wherein the portion of media includes one more genomic fragments shed or secreted from the embryo candidate. A plurality of genomic linker segments and a ligase enzyme is added to the amplification vessel in conditions that catalyze the formation of concatenated genomic fragments containing at least one genomic linker segment and at least one genomic fragment from the isolated embryo candidate. The concatenated genomic fragments are amplified in the amplification vessel. Sequence information is obtained from the concatenated genomic fragments. The sequence information is aligned against a reference genome. Genomic features are identified on the aligned genomic fragment sequences.

Embodiment 13. The method of Embodiment 12, further including: subtracting sequence information related to the genomic linker segment from the concatenated genomic fragment sequence prior to aligning the concatenated genomic fragment sequence to the reference genome.

Embodiment 14. The method of Embodiment 12, further including: blunting the genomic fragment ends using a modified polymerase prior to ligating them to the genomic linker segments.

Embodiment 15. The method of Embodiment 14, wherein the modified polymerase is a Klenow T4 DNA polymerase.

Embodiment 16. The method of Embodiment 12, wherein the ligase enzyme is one of a T3, T4 or T7 prokaryotic DNA ligase.

Embodiment 17. The method of Embodiment 12, wherein the embryo candidate is a human embryo.

Embodiment 18. The method of Embodiment 12, wherein the embryo candidate is a blastocyst.

Embodiment 19. The method of Embodiment 12, wherein the genomic feature is a single nucleotide polymorphism.

Embodiment 20. The method of Embodiment 12, wherein the genomic feature is an indel.

Embodiment 21. The method of Embodiment 12, wherein the genomic feature is an inversion.

Embodiment 22. A system is provided for identifying genomic features in an embryo candidate. The system includes a genomics sequencer, a computing device and a display.

The genomic sequencer is configured to obtain sequence information from concatenated genomic fragments derived from an embryo candidate. The concatenated genomic fragments each contain at least one genomic linker segment and at least one genomic fragment from the embryo candidate.

The computing device is communicatively connected to the genomic sequencer and includes a sequence alignment engine and a genomic features identification engine. The sequence alignment engine is configured to subtract out sequence information related to the genomic linker segment portion of the concatenated genomic fragments and align the genomic fragment sequences to a reference genome. The genomic features identification engine is configured to identify genomic features in the aligned genomic fragment sequences. The display is communicatively connected to the computing device and configured to display a report containing the identified genomic features.

Embodiment 23. The system of Embodiment 22, wherein the genomic feature is a copy number variation.

Embodiment 24. The system of Embodiment 23, wherein the genomic features identification engine is further configured to: normalize a frequency of genomic fragment sequences aligned to each chromosomal position on the reference genome; determine a genomic fragment sequence alignment frequency threshold to make a copy number variation call for each chromosomal position; and make a copy number variation call for each chromosomal positon with genomic fragment sequence alignment frequencies that deviate from the frequency threshold.

Embodiment 25. The system of Embodiment 24, wherein the genomic features identification engine is further configured to apply a circular binary segmentation (CBS) analysis to determine whether the identified deviance from the frequency threshold identified is due to technical bias.

Embodiment 26. The system of Embodiment 24, wherein the normalization is performed using a Spline normalization method.

Embodiment 27. The system of Embodiment 24, wherein a deviance occurs when the frequency of genomic fragment sequences aligned to a chromosomal position is below the frequency threshold.

Embodiment 28. The system of Embodiment 24, wherein a deviance occurs when the frequency of genomic fragment sequences aligned to a chromosomal position is above the frequency threshold.

Embodiment 29. The system of Embodiment 22, wherein the embryo candidate is a human embryo.

Embodiment 30. The system of Embodiment 22, wherein the embryo candidate is a blastocyst.

Embodiment 31. The system of Embodiment 22, wherein the genomic feature is a single nucleotide polymorphism.

Embodiment 32. The system of Embodiment 22, wherein the genomic feature is an indel.

Embodiment 33. The system of Embodiment 22, wherein the genomic feature is an inversion.

Embodiment 34. The system of Embodiment 22, wherein the genomic linker segment sequence is a known sequence.

Embodiment 35. A method is provided for identifying genomic features in a tissue sample is disclosed. Concatenated genomic fragment sequence reads are received containing at least one genomic linker segment sequence and at least one genomic fragment sequence from a tissue sample. The genomic linker segment sequence portion of the concatenated genomic fragment sequence reads is subtracted out. The concatenated genomic fragment sequence reads are aligned (mapped) to a reference genome. Genomic features are identified on the aligned genomic fragment sequences.

Embodiment 36. The method of Embodiment 35, further including: deleting concatenated genomic fragment sequence reads that map to more than one location on a reference genome.

Embodiment 37. The method of Embodiment 35, wherein the genomic feature is a copy number variation.

Embodiment 38. The method of Embodiment 37, further including: normalizing a frequency of genomic fragment sequences aligned to each chromosomal position; determining a genomic fragment sequence alignment frequency threshold to make a copy number variation call for each chromosomal position; and making a copy number variation call for each chromosomal positon with genomic fragment sequence alignment frequencies that deviate from the frequency threshold.

Embodiment 39. The method of Embodiment 38, further including: applying a circular binary segmentation (CBS) analysis to determine whether the identified deviance from the frequency threshold is identified due to technical bias.

Embodiment 40. The method of Embodiment 38, wherein a deviance occurs when the frequency of genomic fragment sequences aligned to a chromosomal position is below the frequency threshold.

Embodiment 41. The method of Embodiment 38, wherein a deviance occurs when the frequency of genomic fragment sequences aligned to a chromosomal position is above the frequency threshold.

Embodiment 42. The method of Embodiment 35, wherein the tissue sample is an embryonic tissue.

Embodiment 43. The method of claim 35, wherein the tissue sample is a blastocyst.

Embodiment 44. The method of claim 35, wherein the genomic feature is a single nucleotide polymorphism.

Embodiment 45. The method of claim 35, wherein the genomic feature is an indel.

Embodiment 46. The method of claim 35, wherein the genomic feature is an inversion.

Embodiment 47. A non-transitory computer-readable medium is provided in which a program is stored for causing a computer to perform a method for identifying genomic features in a tissue sample. Concatenated genomic fragment sequence reads are received containing at least one genomic linker segment sequence and at least one genomic fragment sequence from a tissue sample. The genomic linker segment sequence portion of the concatenated genomic fragment sequence reads are subtracted out. The concatenated genomic fragment sequence reads are aligned (mapped) to a reference genome. Genomic features are identified on the aligned genomic fragment sequences.

Embodiment 48. The method of Embodiment 47, further including: deleting concatenated genomic fragment sequence reads that map to more than one location on a reference genome.

Embodiment 49. The method of Embodiment 47, wherein the genomic feature is a copy number variation.

Embodiment 50. The method of Embodiment 47, wherein the genomic feature is an indel.

Embodiment 51. The method of Embodiment 47, wherein the genomic feature is an inversion.

Embodiment 52. The method of Embodiment 49, further including: normalizing a frequency of genomic fragment sequences aligned to each chromosomal position; determining a genomic fragment sequence alignment frequency threshold to make a copy number variation call for each chromosomal position; and making a copy number variation call for each chromosomal positon with genomic fragment sequence alignment frequencies that deviate from the frequency threshold.

Embodiment 53. The method of Embodiment 52, further including: applying a circular binary segmentation (CBS) analysis to determine whether the identified deviance from the frequency threshold is identified due to technical bias.

Embodiment 54. The method of Embodiment 52, wherein a deviance occurs when the frequency of genomic fragment sequences aligned to a chromosomal position is below the frequency threshold.

Embodiment 55. The method of Embodiment 52, wherein a deviance occurs when the frequency of genomic fragment sequences aligned to a chromosomal position is above the frequency threshold.

Embodiment 56. The method of Embodiment 47, wherein the tissue sample is an embryonic tissue.

Embodiment 57. The method of Embodiment 47, wherein the tissue sample is a blastocyst.

Embodiment 58. The method of Embodiment 47, wherein the genomic feature is a single nucleotide polymorphism.

Embodiment 59. The method of Embodiment 47, wherein the genomic feature is an indel.

Embodiment 60. The method of Embodiment 47, wherein the genomic feature is an inversion.

What is claimed:

1. A method for determining copy number variation in an embryo candidate for in vitro fertilization (IVF) implantation, comprising:
isolating an embryo candidate from a plurality of embryos;
incubating the embryo candidate in media that is substantially free of DNA;
transferring a portion of the media to an amplification vessel, wherein the portion of media includes genomic fragments shed or secreted from the embryo candidate;
adding a plurality of genomic linker segments and ligase enzyme to the amplification vessel in conditions that catalyze the formation of concatenated genomic fragments containing at least one genomic linker segment and at least one genomic fragment from the isolated embryo candidate;
amplifying the concatenated genomic fragments in the amplification vessel;
obtaining sequence information from the amplified concatenated genomic fragments;
aligning the sequence information against a reference genome; and
identifying copy number variations in the embryo candidate when a frequency of genomic fragment sequence reads aligned to a chromosomal position on the reference genome deviates from a frequency threshold.

2. The method of claim 1, further including:
subtracting sequence information related to the genomic linker segment from the concatenated genomic fragment sequence prior to aligning the concatenated genomic fragment sequence to the reference genome.

3. The method for claim 2, further including:
normalizing the frequency of genomic fragment sequence reads aligned to each chromosomal position; and
determining a frequency threshold for each chromosomal position.

4. The method of claim 3, further including:
applying a circular binary segmentation (CBS) analysis to determine whether the identified deviance from the frequency threshold identified is due to technical bias.

5. The method of claim 3, wherein the normalization is performed using a Spline normalization method.

6. The method of claim 1, further including:
blunting the genomic fragment ends using a modified polymerase prior to ligating them to the genomic linker segments.

7. The method of claim 6, wherein the modified polymerase is a Klenow T4 DNA polymerase.

8. The method of claim 1, wherein the ligase enzyme is one of a T3, T4 or T7 prokaryotic DNA ligase.

9. The method of claim 1, wherein the embryo candidate is a human embryo.

10. The method of claim 1, wherein the embryo candidate is a blastocyst.

11. The method of claim 1, wherein the frequency threshold is a frequency of genomic fragment reads that map to a normal chromosome.

12. A method for identifying genomic features in an embryo candidate, comprising:
isolating an embryo candidate from a plurality of embryo candidates;
incubating the embryo candidate in media that is substantially free of DNA;
transferring a portion of the media to an amplification vessel, wherein the portion of media includes one more genomic fragments shed or secreted from the embryo candidate;
adding a plurality of genomic linker segments and a ligase enzyme to the amplification vessel in conditions that catalyze the formation of concatenated genomic fragments containing at least one genomic linker segment and at least one genomic fragment from the isolated embryo candidate;
amplifying the concatenated genomic fragments in the amplification vessel;
obtaining sequence information from the concatenated genomic fragments;
aligning the sequence information against a reference genome; and
identifying genomic features on the aligned genomic fragment sequences.

13. The method of claim 12, further including:
subtracting sequence information related to the genomic linker segment from the concatenated genomic fragment sequence prior to aligning the concatenated genomic fragment sequence to the reference genome.

14. The method of claim 12, further including:
blunting the genomic fragment ends using a modified polymerase prior to ligating them to the genomic linker segments.

15. The method of claim 14, wherein the modified polymerase is a Klenow T4 DNA polymerase.

16. The method of claim 12, wherein the ligase enzyme is one of a T3, T4 or T7 prokaryotic DNA ligase.

17. The method of claim 12, wherein the embryo candidate is a human embryo.

18. The method of claim 12, wherein the embryo candidate is a blastocyst.

19. The method of claim 12, wherein the genomic feature is a single nucleotide polymorphism.

20. The method of claim 12, wherein the genomic feature is an indel.

21. The method of claim 12, wherein the genomic feature is an inversion.

22. A system for identifying genomic features in an embryo candidate, comprising:
a genomic sequencer configured to obtain sequence information from concatenated genomic fragments derived from an embryo candidate, wherein the concatenated genomic fragments each contain at least one genomic linker segment and at least one genomic fragment from the embryo candidate;
a computing device communicatively connected to the genomic sequencer comprising,
a sequence alignment engine configured to subtract out sequence information related to the genomic linker segment portion of the concatenated genomic fragments and align the genomic fragment sequences to a reference genome, and
a genomic features identification engine configured to identify genomic features in the aligned genomic fragment sequences; and
a display communicatively connected to the computing device and configured to display a report containing the identified genomic features.

23. The system of claim 22, wherein the genomic feature is a copy number variation.

24. The system of claim 23, wherein the genomic features identification engine is further configured to,
normalize a frequency of genomic fragment sequences aligned to each chromosomal position on the reference genome;
determine a genomic fragment sequence alignment frequency threshold to make a copy number variation call for each chromosomal position; and
make a copy number variation call for each chromosomal positon with genomic fragment sequence alignment frequencies that deviate from the frequency threshold.

25. The system of 24, wherein the genomic features identification engine is further configured to,
apply a circular binary segmentation (CBS) analysis to determine whether the identified deviance from the frequency threshold identified is due to technical bias.

26. The system of claim 24, wherein the normalization is performed using a Spline normalization method.

27. The system of claim 24, wherein a deviance occurs when the frequency of genomic fragment sequences aligned to a chromosomal position is below the frequency threshold.

28. The system of claim 24, wherein a deviance occurs when the frequency of genomic fragment sequences aligned to a chromosomal position is above the frequency threshold.

29. The system of claim 22, wherein the embryo candidate is a human embryo.

30. The system of claim 22, wherein the embryo candidate is a blastocyst.

31. The system of claim 22, wherein the genomic feature is a single nucleotide polymorphism.

32. The system of claim 22, wherein the genomic feature is an indel.

33. The system of claim 22, wherein the genomic feature is an inversion.

34. The system of claim 22, wherein the genomic linker segment sequence is a known sequence.

35. A method for identifying genomic features in a tissue sample, comprising
receiving concatenated genomic fragment sequence reads containing at least one genomic linker segment sequence and at least one genomic fragment sequence from a tissue sample;
subtracting out the genomic linker segment sequence portion of the concatenated genomic fragment sequence reads;
aligning the concatenated genomic fragment sequence reads to a reference genome; and
identifying genomic features on the aligned genomic fragment sequences.

36. The method of claim 35, further including:
deleting concatenated genomic fragment sequence reads that map to more than one location on a reference genome.

37. The method of claim 35, wherein the genomic feature is a copy number variation.

38. The method of claim 37, further including:
normalizing a frequency of genomic fragment sequences aligned to each chromosomal position;
determining a genomic fragment sequence alignment frequency threshold to make a copy number variation call for each chromosomal position; and
making a copy number variation call for each chromosomal positon with genomic fragment sequence alignment frequencies that deviate from the frequency threshold.

39. The method of claim 38, further including:
applying a circular binary segmentation (CBS) analysis to determine whether the identified deviance from the frequency threshold is identified due to technical bias.

40. The method of claim 38, wherein a deviance occurs when the frequency of genomic fragment sequences aligned to a chromosomal position is below the frequency threshold.

41. The method of claim 38, wherein a deviance occurs when the frequency of genomic fragment sequences aligned to a chromosomal position is above the frequency threshold.

42. The method of claim 35, wherein the tissue sample is an embryonic tissue.

43. The method of claim 35, wherein the tissue sample is a blastocyst.

44. The method of claim 35, wherein the genomic feature is a single nucleotide polymorphism.

45. The method of claim 35, wherein the genomic feature is an indel.

46. The method of claim 35, wherein the genomic feature is an inversion.

47. A non-transitory computer-readable medium in which a program is stored for causing a computer to perform a method for identifying genomic features in a tissue sample, the method comprising:
receiving concatenated genomic fragment sequence reads containing at least one genomic linker segment sequence and at least one genomic fragment sequence from a tissue sample;
subtracting out the genomic linker segment sequence portion of the concatenated genomic fragment sequence reads;
aligning the concatenated genomic fragment sequence reads to a reference genome; and
identifying genomic features on the aligned genomic fragment sequences.

48. The method of claim 47, further including:
deleting concatenated genomic fragment sequence reads that map to more than one location on a reference genome.

49. The method of claim 47, wherein the genomic feature is a copy number variation.

50. The method of claim 47, wherein the genomic feature is an indel.

51. The method of claim 47, wherein the genomic feature is an inversion.

52. The method of claim 49, further including:
normalizing a frequency of genomic fragment sequences aligned to each chromosomal position;
determining a genomic fragment sequence alignment frequency threshold to make a copy number variation call for each chromosomal position; and
making a copy number variation call for each chromosomal positon with genomic fragment sequence alignment frequencies that deviate from the frequency threshold.

53. The method of claim 52, further including:
applying a circular binary segmentation (CBS) analysis to determine whether the identified deviance from the frequency threshold is identified due to technical bias.

54. The method of claim 52, wherein a deviance occurs when the frequency of genomic fragment sequences aligned to a chromosomal position is below the frequency threshold.

55. The method of claim 52, wherein a deviance occurs when the frequency of genomic fragment sequences aligned to a chromosomal position is above the frequency threshold.

56. The method of claim 47, wherein the tissue sample is an embryonic tissue.

57. The method of claim 47, wherein the tissue sample is a blastocyst.

58. The method of claim 47, wherein the genomic feature is a single nucleotide polymorphism.

59. The method of claim 47, wherein the genomic feature is an indel.

60. The method of claim 47, wherein the genomic feature is an inversion.
